SEQUENCE LISTING (3 sequences)

SEQ ID NO: 1 - DNA, 22 bp, Artificial sequence
  Oligonucleotide
ggttgtggatgccgataccggg

SEQ ID NO: 2 - DNA, 22 bp, Artificial sequence
  Oligonucleotide
gctatgtctccagtatgtagcc

SEQ ID NO: 3 - DNA, 5777 bp, Artificial sequence
  Plasmid
nasncrrmatwachckrmtnndcmntatnwangthntynchckaagcttactccccatccccctgttgacaattaatcatcggctcgtataatgtgtggaattgtgagcggataacaatttcacacaggaaacaggatccaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatcctccgaggcctcgatcgaggtcgacgaattcggtaaatttatggttggttatgaaatgctggcagagacccagcgagacctgaccgcagaacaggcagcagagcgtttgcgcgcagtcagcgatatccattttcgcgaatccggagtgtaagaaatgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctgaacgaaactcccgcactggcacccgatggtcagccgtaccgactgttctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctggcgaatcctctgaccagccagaaaacgacctttctgtggtgaaaccggatgctgcaattcagagcgccagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggcc

(19) United States
(12) Patent Application Publication
(10) Pub. No.: US 2004/0185457 A1
(43) Pub. Date: Sep. 23, 2004

(54) Method for detecting dna polymerisation

(76) Inventors: James Augustus Henry Murray, Cambridge (GB); Laurence Carlo Tisi, Cambridge (GB)

Correspondence Address: NIXON & VANDERHYE, PC, 1100 N GLEBE ROAD, 8TH FLOOR, ARLINGTON, VA, 22201-4714, US

(21) Appl. No.: 10/467,781

(22) PCT Filed: Feb. 13, 2002
(86) PCT No.: PCT/GB02/00648

(30) Foreign Application Priority Data
Feb. 14, 2001 (GB) 0103622.7

(51) Int. Cl. C12Q 1/68; C12Q 1/66

(52) U.S. Cl. 435/6; 435/8

(57) ABSTRACT

A method for determining the extent of a processive nucleic acid polymerase reaction producing pyrophosphate can be conducted, in the presence of all components necessary for the progression of nucleic acid synthesis, wherein the components comprise a substrate for the nucleic acid polymerase which is either dATP or a dATP analogue. The method includes a pyrophosphate assay comprising the steps of conversion of the pyrophosphate to ATP and detection of light produced by the bioluminescence reaction of a luciferase with ATP, wherein one or both of the following apply: a) the luciferase reacts with ATP and the substrate, such that the spectral overlap is reduced relative to the spectral overlap between the outputs of reaction between wild-type Photinus pyralis luciferase with ATP and dATP respectively; and b) the luciferase reacts with the substrate to give a reduced bioluminescence relative to that produced by the reaction of wild-type Photinus pyralis luciferase with dATP.

### FIELD OF THE INVENTION

The present invention relates to methods for the analysis of samples produced by the in vitro polymerisation of nucleic acids, preferably the products of DNA polymerisation, particularly the products of a polymerase chain reaction. This invention also relates to kits utilising such methods.

### BACKGROUND OF THE INVENTION

In vitro DNA polymerisation is commonly in the form of the polymerase chain reaction (PCR), a ubiquitous technique in molecular biology. PCR allows the amplification (and modification) of precisely defined DNA sequences from, if necessary, very small quantities of DNA.

Commonly, PCR is analysed using gel electrophoresis (either agarose or acrylamide-based) using a fluorescent dye (such as ethidium bromide) to stain for the presence of DNA. This method can be used to indicate the number, amount and size of amplified PCR products. However, the preparation, running and analysis of PCR reactions using gel electrophoresis requires extensive manual intervention and hazardous reagents and is time-consuming (typically taking around 1 hour in total). In addition, multiple PCR cycles (typically 30) are required to produce detectable product.

Recent methods do not use an electrophoresis step in the analysis of PCR. Such techniques work by monitoring a change in fluorescence that is associated with the accumulation of a PCR product by some means.

Means to monitor amplification of DNA during PCR using double-stranded DNA-binding dyes, specific hybridisation probes containing donor and acceptor fluorophores or PCR primer probes containing donor and acceptor fluorophores, are described in WO-A-97/444486, WO-A-99/42611 and U.S. Pat. No. 6,174,670.

Such methods have the benefit over gel electrophoresis of making it possible to follow PCR without liquid sampling, so allowing a reaction to be followed in real time. This allows PCR product to be detected quickly, with fewer cycles, and gives direct information on annealing/melting kinetics.

However, the fluorescent techniques have significant drawbacks. In particular, specialised equipment and reagents are required. Normally a computer-driven integrated thermocycler-fluorimeter is required as the methods often follow PCR in real-time rather than being employed for end-point analyses. As a result, the accessibility (cost), and portability of such systems is compromised. Since detection is carried out within the PCR instrument, such methods are only available to suitably equipped laboratories.

In addition, the application of fluorescence-based systems may be hampered by the limited capacity of equipment and its high cost. Finally, costs of fluorescent reagents, particularly fluorescently-labeled primers, are high and sample preparation can be cumbersome.

A characteristic of DNA and RNA polymerases is the fact that they release the compound pyrophosphate (PPi) each time they incorporate a new base into the growing DNA/RNA molecule. The reaction may be shown as, for a polymer of length n:

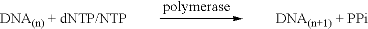

A sensitive assay for pyrophosphate is known as the Enzymatic Luminometric Inorganic Pyrophosphate Detection Assay (ELIDA); see Nyren et al, Anal. Biochem. 220: (1) 39-45 (1994). This assay has two steps: (1) conversion of pyrophosphate (PPi) to ATP by the enzyme ATP sulphurylase, and (2) utilisation of the ATP to produce light in the presence of luciferase and oxygen, catalysed by firefly luciferase (known in the art):

ELIDA has been applied, to follow the incorporation of single nucleotides into a DNA molecule by a polymerase; see U.S. Pat. No. 5,534,424, U.S. Pat. No. 5,498,523, WO-A-98/28440 and WO-A-98/13523. ELIDA-based methods are available for identifying single base polymorphisms as well as sequencing short regions of DNA. The approaches are iterative in nature, and detect the incorporation of individual nucleotides. By definition, such applications are not detecting processive nucleic acid polymerase activity.

One of the components of a polymerase reaction is the deoxynucleotide dATP which is also a substrate for luciferase. Thus an intrinsic problem encountered in a pyrophosphate assay of a polymerisation reaction is the spectral interference resulting from the reaction of luciferase with dATP.

A method for detecting a DNA polymerisation reaction is disclosed in WO-A-92/16654 and the article by Tabary et al., J. Immunological Methods, 156 (1992) 55-60. This method does not directly address the problem of dATP interference, but attempts to diminish the effects of interference using a lengthy stepwise assay involving the multiple addition of reagents.

### SUMMARY OF THE INVENTION

According to the present invention, a method for determining the extent of a processive nucleic acid polymerase reaction producing pyrophosphate, in the presence of all components necessary for the progression of nucleic acid synthesis, wherein the components comprise a substrate for the nucleic acid polymerase which is either dATP or a dATP analogue, includes a pyrophosphate assay comprising the steps of conversion of the pyrophosphate to ATP and detection of light produced by the bioluminescence reaction of a luciferase with ATP, wherein one or both of the following apply:

a) the luciferase reacts with ATP and the substrate, such that the spectral overlap is reduced relative to the spectral overlap between the outputs of reaction between wild-type Photinus pyralis luciferase with ATP and dATP respectively; and

b) the luciferase reacts with the substrate to give a reduced bioluminescence relative to that produced by the reaction of wild-type Photinus pyralis luciferase with dATP.

The invention provides simple, cheap and rapid alternatives to both gel electrophoresis-based and fluorescence-based techniques. It can be used to distinguish whether or not a PCR reaction has taken place (and/or quantify the extent of a PCR reaction), using simple, cheap hardware requirements, with the possibility of portability and miniaturisation and easy integration into high throughput systems. The method can provide improved signal stability and improved signal:background ratio.

### DESCRIPTION OF THE INVENTION

The invention provides a method to assay in vitro enzymatic synthesis of nucleic acid and to quantify the extent to which the nucleic acid has been polymerised de novo. The invention comprises the detection of pyrophosphate, produced as a consequence of nucleic acid polymerisation.

Preferably, the assay is used to analyse the results from the polymerase chain reaction (PCR), where DNA molecules are amplified in vitro. The assay can be carried out in the presence of all the components of the polymerisation reaction, including the nucleotides that are required.

In one embodiment, the invention comprises the steps of

(1) converting pyrophosphate into ATP using the enzyme ATP sulphurylase

(2) detecting the ATP produced, preferably by an enzyme that produces light in the presence of ATP and an appropriate substrate, more preferably firefly luciferase, wherein the ATP produced as a consequence of step (1) can be distinguished from dATP or analogues which may be utilised by the polymerase enzyme.

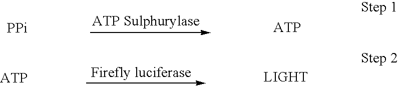

The invention allows the ATP produced in step 1 to be distinguished from nucleotides present as a substrate for the polymerase enzyme. These methods comprise the alternatives of

(a) optical methods to distinguish the wavelength of light produced from ATP in step 2, from the light produced by enzyme action on the nucleotides present in the DNA polymerisation reaction buffer

(b) utilising nucleotides in the polymerisation reaction that are not effective substrates for the enzyme used in step 2

(c) utilising enzyme variants in step 2 with reduced utilisation of dATP as a substrate.

The invention comprises the detection of pyrophosphate as a means of determining the extent of a processive nucleic acid polymerase enzyme. “Processive” as used herein refers to an enzyme operating under conditions where more than one nucleotide addition cycle can be carried out without further additions to or manipulation of the buffer components.

This invention relates to detecting the pyrophosphate produced by a nucleic acid polymerase enzyme, preferably in the presence of the components needed by the enzyme. More preferably the pyrophosphate is first converted to ATP (e.g. by ATP sulphurylase). The ATP is then detected, preferably, by a bioluminescence reaction, more preferably using a beetle luciferase, most preferably firefly luciferase.

The novel assay is performed subsequent to processive polymerisation of a nucleic acid molecule, preferably a DNA molecule, more preferably after application of the polymerase chain reaction. Further, the assay is performed directly on the reaction mixture used for the enzymatic DNA synthesis in the presence of all the components necessary for DNA synthesis.

The components of an enzymatic DNA synthesis reaction will typically contain a mixture of buffer, MgCl₂, dATP, dCTP, dGTP, dTTP, primer(s), a DNA template and a polymerase. Such a mixture would interfere with an ELIDA assay for pyrophosphate since, in particular, the dATP reacts directly with firefly luciferase, producing light. Given that the concentration of dATP typically used in such reactions is high (e.g. 0.2 mM), light production from dATP reacting directly with luciferase will contribute significantly to the light detected so confounding the assay for pyrophosphate, particularly at low pyrophosphate concentrations.

The invention embodies methods to assay pyrophosphate in the presence of the reagents required for enzymatic DNA synthesis without undue interference from light not produced as a consequence of the production of pyrophosphate. As such, considering the first equation seen above, the invention can be used to assess the extent to which DNA polymerisation has been occurring in a sample, preferably a PCR reaction. The invention can thus be used to assess whether or not a PCR reaction has produced any PCR product, and also to quantify how much PCR product has been produced. In many applications (e.g. rapid detection of DNA associated with pathogens) where the PCR conditions are already sufficiently optimised, the only information required to establish that the target DNA sequence was present in the sample is the occurrence of DNA polymerisation.

In one embodiment of the invention, wavelength-specific light detection may be employed to differentiate between light produced from ATP (and hence derived from pyrophosphate) and light from dATP. Firefly luciferase reacts with ATP to give yellow-green light, whereas it reacts with dATP to give red light. As a result, where a sample contains both ATP and dATP, the extent to which the light produced in the presence of luciferase is from dATP can be assessed by either employing suitable filters or measuring light emission at wavelengths where dATP contributes little to the recorded signal compared to ATP. Thus the light derived from dATP reacting directly with luciferase can be sufficiently minimised to make pyrophosphate detection in a PCR reaction feasible, even at low pyrophosphate concentration.

In another embodiment of the invention, dATP is replaced by d-α-SATP. d-α-SATP is an approximately one-hundred-fold poorer substrate for luciferase than dATP, such that there is very little light production from the direct reaction of d-α-SATP with luciferase. Nevertheless, d-α-SATP is a suitable substrate for DNA amplification reactions. It is known that d-α-SATP can be incorporated into a DNA strand by DNA polymerases. It has now been found that DNA that has incorporated d-α-SATP (now containing sulphur atoms) can act as a template to direct further DNA synthesis; this is a prerequisite for PCR. PCR reactions using d-α-SATP are efficient and, due to the lack of dATP, they do not intrinsically cause significant light emission when added to ELIDA reagent. Thus, when using d-α-SATP in a PCR reaction, the resulting sample will only emit significant light if pyrophosphate has been generated, e.g. if a PCR product has been amplified. It will be evident to a person skilled in the art that nucleotides other than d-α-SATP which are substrates for PCR but whose reaction with firefly luciferase demonstrates a lower specific activity than with dATP, could also be utilised in this context.

Another embodiment of the invention is the application of mutant firefly luciferases or beetle luciferase variants with either a greater difference in the colour of bioluminescence using dATP compared to ATP or a different affinity for either dATP or ATP. Mutant luciferases where the difference between the wavelength of maximal luminescence with dATP and ATP larger than wild-type enzyme can be employed to improve the discrimination between light produced from ATP as opposed to dATP. Further, mutants where dATP is a poorer substrate for light emission relative to ATP, may be similarly employed. Within this embodiment, any mutant luciferase can be used which is more discriminating with respect to reacting with ATP over other nucleotides, either in activity or wavelength of light produced. Methods for obtaining or identifying such mutant luciferases are known in the art (e.g. WO-A-96/22376 and WO-A-99/14336).

It is a feature of the invention that pyrophosphate from DNA synthesis can be detected when the DNA which has been synthesised would be undetectable by gel electrophoresis, resulting in increased sensitivity and reduced amplification time in PCR assays. In addition, the amount of pyrophosphate detected is proportional to the amount of DNA synthesised under appropriate conditions. Hence the detection of pyrophosphate can be used to quantitate a PCR reaction.

A further feature of the invention is the utilisation of pyrophosphate assay components that can withstand the conditions of a PCR reaction. The pyrophosphate assay components may be stabilised by lyophilisation or by the presence of stabilising factors, and/or may be thermostable, in particular thermostable ATP sulphurylase amd thermostable luciferase. The nucleic acid polymerase may also be thermostable.

Analysis of a PCR sample simply requires the mixing of some PCR sample with ELIDA reagent. Light emission from the mixture is rapid and of sufficient magnitude that complex light-detection hardware is not necessary: a CCD camera or even photographic film can be used to analyse the result.

Further, the light signal from the ELIDA reaction is stable over a few minutes, giving a useful window of time in which the assay can be measured. This means, for example, that a PCR reaction done on a multiwell-plate format, requires only ELIDA reagent added to all the wells (which is easily done simultaneously with simple liquid handling devices) and placed under a CCD camera for a few seconds, to give a complete result. The potential for very high throughput analysis is therefore clear: liquid handling is simple, as is the hardware, and hundreds of samples may be easily analysed simultaneously.

Further, the simplicity of the assay offers far greater accessibility and portability of PCR and other technology. In particular, this assay may be easily integrated into miniature “liquid” circuits performing PCR reactions. Indeed, the assay offers the benefit of not requiring an in-built fluorimeter as would methods using fluorescent oligos: a simple photomultiplyer or photographic film is sufficient to analyse the results from the invention disclosed.

The following Examples illustrate the invention.

### EXAMPLE 1

### Use of Filters to Remove Light Contributed from dATP Stimulated Bioluminescence

Pyrophosphate assay buffer (PAB) was made up as follows:

| 0.1 | M | Tris-acetate (pH7.75) | Sigma |
|---|---|---|---|
| 2 | mM | EDTA | ″ |
| 10 | mM | Magnesium Acetate | ″ |
| 0.1% | | Bovine serum albumin | ″ |
| 5 | μM | Adenosine 5′ phosphosulphate | ″ |
| 0.4 | mg/ml | Polyvinylpyrrolidone (360,000) | ″ |
| 0.3 | U/ml | ATP Sulphurylase | ″ |
| 100 | mg/ml | D-luciferin | Europa |
| 5.5 × 10⁸ | light units | Photinus pyralis luciferase | Promega |
| 1 | mM | Dithiothreotol | Melford |

Solutions of sodium pyrophosphate (Sigma) were made up freshly and stored shaded on ice.

50 μl of PAB was aliquoted into two wells of a 96 well plate. To one sample was added 1 μl of 0.1 mM sodium pyrophosphate; to the other was added 1 μl of 2 mM dATP. The resulting light emission was observed using a Syngene light-box with a black and white CCD camera integrating the signal over 5 seconds. Whilst the sample where pyrophosphate was added is far brighter, there is significant light emission from the dATP sample. The amount of dATP added is equivalent to ⅕ the amount used in a standard 50 μl PCR reaction. Hence the contribution from the dATP to the bioluminescence is significant.

A green filter was placed in front of the lens of the CCD camera and the sample above was observed once more. It could be clearly seen that the presence of the green filter markedly reduced the signal of the dATP sample relative to the pyrophosphate sample. This was by virtue of the light emission from the dATP sample having a maximal light emission at 620 nm whereas the emission from ATP is at a maximum at 550 nm (at pH 7.8) hence the green filter blocked more of the light from the dATP sample.

### EXAMPLE 2

### PCR using the dATP Analogue d-α-SATP

A 50 μl PCR reaction contained the following:

| | | | | |
|---|---|---|---|---|
| 5 μl | Tris-HCl | 100 | mM | Sigma |
| 4 μl | Magnesium Chloride | 50 | mM | Gibco |
| 5 μl | d-α-SATP | 2 | mM | Glen research |
| 5 μl | dCTP | 2 | mM | Pharmacia |
| 5 μl | dGTP | 2 | mM | Pharmacia |
| 5 μl | dTTP | 2 | mM | Pharmacia |
| 1 μl | Test plasmid pPw601a | 0.5 | ng/μl | |
| 1.25 μl | Primer 1 | 10 | μM | |
| 1.25 μl | Primer 2 | 10 | μM | |
| 0.5 μl | Taq polymerase | 5 | U/μl | Roche |
| 19 μl | Milli-Q water | | | |

The sequences of primers 1 and 2 and the test plasmid are given in SEQ ID Nos. 1, 2 and 3, respectively. The size of the expected PCR product was 158 bp.

5 μl of Eurogentec “smart-ladder” molecular weight marker was used in gel electrophoresis. 2 μl of 10 mg/ml ethidium bromide was added to agarose gels and gel running buffer.

Taq polymerase was added to the sample after heating to 95° for 30 seconds. The following cycling parameters were implemented on a Perkin-Elmer GeneAmp PCR system 2400.

| | |
|---|---|
| 95° C. | 30 sec |

(addition of Taq polymerase)

| | | | |
|---|---|---|---|
| 95° C. | 30 sec | | |
| 95° C. | 30 sec | | |
| 55° C. | 30 sec | {close oversize brace} | 30 cycles |
| 72° C. | 5 sec | | |
| 72° C. | 20 sec | | |

10 μl of the resulting PCR reaction was loaded onto a 10% agarose gel containing ethidium bromide alongside a molecular weight marker. The gel was run at 60V for 50 minutes and then visualised under a transilluminator. It could be seen that a PCR product is generated using d-α-SATP instead of dATP. Under the same conditions, if dATP is used instead but at 1/10th the usual concentration of 0.2 mM, no PCR product is detected. This shows that the PCR product gained using d-α-SATP is not simply the result of contamination by dATP as the d-α-SATP was over 98% pure; hence a PCR product could not have occurred by virtue of contaminating dATP.

### EXAMPLE 3

### Dectection of Pyrophosphate from a PCR Reaction using d-α-SATP

Three PCR reactions were set up as in Example 2. One of the samples was not subjected to thermocycling, the remainder were thermocycled but one did not have Taq added. The expected result was that only the sample thermocycled with Taq would produce a PCR product. This was confirmed by gel electrophoresis. 5 μl of each PCR reactions was added to 25 μl of ELIDA buffer in a 96 well plate. Light emission from the samples was measured using a CCD camera, and it was demonstrated that the light emission from the samples, on assaying for pyrophosphate, clearly corresponds to the gel result.

### EXAMPLE 4

### Dectection of Pyrophosphate from a PCR Reaction using dATP

PCR reactions were set up as in Example 2, except that dATP was used instead of d-α-SATP, i.e. standard PCR reaction conditions were used. Two samples were prepared, one with the correct DNA template present (with respect to the primers used) and one where the correct DNA template was not present. The expected result was that only the sample with the correct template would produce PCR product. Having run the PCR reaction, the samples were analysed by gel electrophoresis. 5 μl of each PCR reaction was then added to 25 μl of ELIDA assay reagent and the light emitted detected with a CCD camera fitted with a green filter, as in Example 1. The detected light emission corresponds with that of the gel electrophoresis, such that the ELIDA assay is able to indicate correctly which sample has successfully produced PCR product.

### EXAMPLE 5

### Quantification and Sensitivity of PCR Product Detection

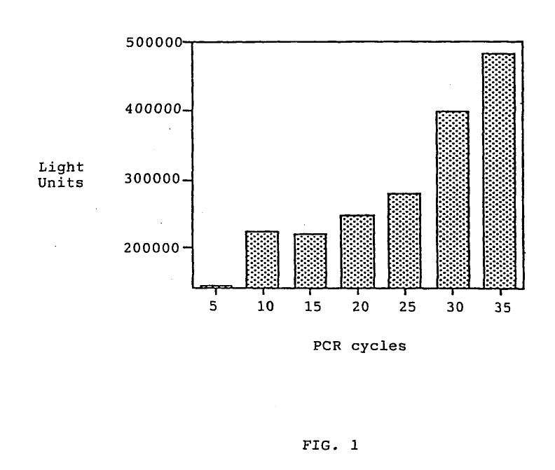

Seven PCR samples were prepared as in Example 2 (i.e. using d-a-SATP). During thermocycling, one of the tubes was removed and placed on ice every 5 cycles up to 35 cycles of the PCR reaction. The production of PCR product was analysed using gel electrophoresis. 5 μl of each of the samples was added to 25 μl of ELIDA buffer and the light emitted was recorded with a CCD camera. FIG. 1 (a graph of light against the number of PCR cycles) shows that, not only does the light emitted from the ELIDA assays increase with cycle number, but that light emission increases before PCR product can be detected on the gel.

### EXAMPLE 6

### Stability of Light Emission from ELIDA Assays of PCR Reactions Containg d-α-SATP Compared to dATP

PCR reactions were set up and run as in Example 5. The analysis of each PCR sample was initiated by mixing 1 μl of the PCR reaction with 20 μl of ELIDA assay buffer. The light emitted from the sample was then measured with a Labsystems Luminoscan Ascent luminometer in a series of four consecutive light readings over a period of 1 minute. Each light reading represented the light emission from the sample integrated over a ten-second interval (using the machine's default PMT voltage).

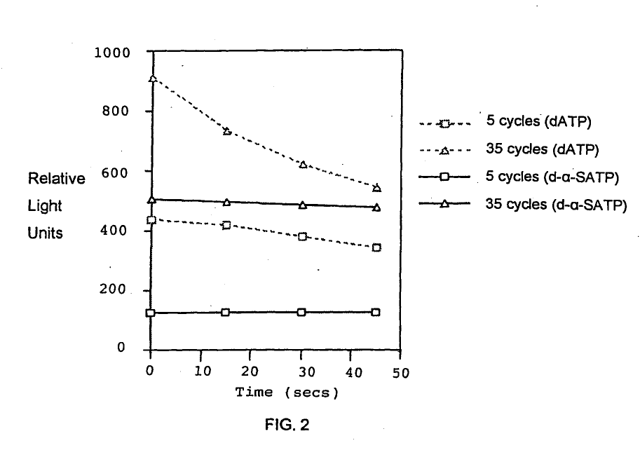

FIG. 2 (a graph of relative light units against time) demonstrates the magnitude of the light emission over four consecutive light readings from ELIDA assays of PCR reactions run for 5 and 35 cycles with either dATP or d-α-SATP. PCR reactions run with dATP show significant decay in light emission over time. This effect becomes greater with the number of PCR cycles. As a result, the differential between the light emitted between 5 and 35 cycles significantly decreases. Conversely, using d-α-SATP, the light emission over time is close to constant for both the 5 and 35 cycle samples. As a result, the magnitude of the differential between the readings for 5 and 35 cycles over the four readings taken, is almost constant (only 5% difference).

### EXAMPLE 7

### Background to Signal Ratio in ELIDA Assays of PCR Reactions Containing d-α-SATP Compared to dATP

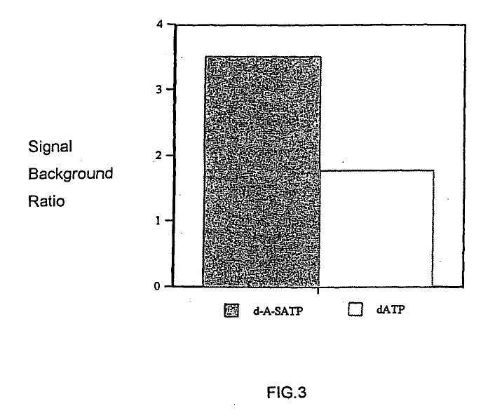

PCR reactions were set up as in Example 2 except that only 0.5 pg of the test plasmid pPw601 a was used. Further, the PCR reactions contained either d-α-SATP or dATP. The light emission from an ELIDA assay of unreacted PCR mixes containing either d-α-SATP or dATP was measured as in Example 6. A further ELIDA assay was performed on the same samples after they had completed 30 cycles of a PCR reaction (and had produced the same amount of amplicon as judged by agarose gel analysis). The ratio of the light emission from the unreacted PCR mix to the reacted PCR mix is shown in FIG. 3 (showing signal:background ratio). It can be seen that using d-α-SATP considerably improves the signal:background ratio. This is due to the fact that d-α-SATP does not react with luciferase to the same degree as dATP.

1. A method for determining the extent of a processive nucleic acid polymerase reaction producing pyrophosphate, in the presence of all components necessary for the progression of nucleic acid synthesis, wherein the components comprise a substrate for the nucleic acid polymerase which is either dATP or a dATP analogue, wherein the method includes a pyrophosphate assay comprising the steps of conversion of the pyrophosphate to ATP and detection of light produced by the bioluminescence reaction of a luciferase with ATP, wherein one or both of the following apply:
a) the luciferase reacts with ATP and the substrate, such that the spectral overlap is reduced relative to the spectral overlap between the outputs of reaction between wild-type. Photinus pyralis luciferase with ATP and dATP respectively; and
b) the luciferase reacts with the substrate to give a reduced bioluminescence relative to that produced by the reaction of wild-type Photinus pyralis luciferase with dATP.

2. A method according to claim 1, wherein the substrate is dATP.

3. A method according to claim 1, wherein the substrate is a dATP analogue.

4. A method according to claim 3, wherein the dATP analogue is d-α-S-ATP.

5. A method according to any preceding claim, wherein the luciferase is a variant or mutant of a coleopteran luciferase.

6. A method according to claim 5, wherein the luciferase is a variant or mutant of Photinus pyralis luciferase.

7. A method according to claim 3 or claim 4, wherein the luciferase is wild-type Photinus pyralis luciferase.

8. A method according to any preceding claim, wherein light produced by the bioluminescence reaction is detected using wavelength-specific light detection.

9. A method according to any preceding claim, wherein a light filter is used in the pyrophosphate assay.

10. A method according to any preceding claim, wherein the luciferase is a thermostable luciferase.

11. A method according to any preceding claim, wherein the nucleic acid polymerase is a DNA polymerase.

12. A method according to claim 11, wherein the nucleic acid polymerase is thermostable.

13. A method according to any preceding claim, wherein the conversion comprises the addition of ATP sulphurylase.

14. A method according to claim 13, wherein the ATP sulphurylase is thermostable.

15. A method according to any preceding claim, wherein a component of the pyrophosphate assay is immobilised.

16. A method according to any preceding claim, wherein a component of the pyrophosphate assay is stabilised by lyophilisation or by the presence of stabilising factors.

17. A kit suitable for use in a method defined in any preceding claim, comprising containers respectively containing
a) buffered mixture of nucleic acid polymerase, a source of Mg, and deoxynucleotides, wherein the deoxynucleotides comprise a substrate as defined in claim 1; and
b) a luciferase, luciferin and ATP sulphurylase.
